(19) United States
(12) Patent Application Publication
(10) Pub. No.: US 2020/0178904 A1
(43) Pub. Date: Jun. 11, 2020

(54) APPARATUS, METHODS AND SYSTEMS FOR DISPLAYING INTRALUMINAL IMAGES

(71) Applicant: Canon U.S.A., Inc., Melville, NY (US)

(72) Inventor: Jeffrey Yutien Chen, Winchester, MA (US)

(21) Appl. No.: 16/701,679

(22) Filed: Dec. 3, 2019

Related U.S. Application Data
(60) Provisional application No. 62/778,166, filed on Dec. 11, 2018.

(51) Int. Cl. A61B 5/00; A61B 1/00; A61B 1/005

(52) U.S. Cl. CPC A61B 5/7267; A61B 1/00165; A61B 1/00009; A61B 5/0066; A61B 1/005; A61B 1/00172

(57) ABSTRACT

Apparatus, methods and systems for displaying continuous images of a lumen while accurately depicting the curvature of the lumen, as well as evaluating and diagnosing biological objects, such as, but not limited to, gastro-intestinal, pulmonary and/or intravascular applications, which may be obtained via one or more instruments, such as, but not limited to, probes, catheters, endoscopes, capsules, and needles (e.g., a biopsy needle).

### CROSS REFERENCE TO RELATED PATENT APPLICATIONS

This application claims priority from U.S. Provisional Patent Application No. 62/778,166 filed on Dec. 11, 2018, in the United States Patent and Trademark Office, the disclosure of which is incorporated herein in its entirety by reference.

### FIELD OF THE DISCLOSURE

The present disclosure relates generally to apparatus, methods and systems for imaging cross-sections of a lumen. More particularly, the present disclosure is directed toward apparatus, methods and systems for displaying continuous images of a lumen while accurately depicting the curvature of the lumen. In addition, the subject disclosure is relevant in evaluating and diagnosing biological objects, such as, but not limited to, gastro-intestinal, pulmonary and/or intravascular applications, which may be obtained via one or more instruments, such as, but not limited to, probes, catheters, endoscopes, capsules, and needles (e.g., a biopsy needle).

### BACKGROUND OF THE DISCLOSURE

Percutaneous coronary intervention (PCI) has improved since the introduction of intravascular imaging (IVI) modalities such as intravascular ultrasound (IVUS) and optical coherence tomography (OCT). IVI modalities provide cross-sectional imaging of coronary arteries with precise lesion information. While OCT allows for imaging using coherent light to capture micrometer-resolution, two- and three-dimensional images from within optical scattering media (e.g., biological tissue). Three-dimensional imaging is accomplished by recording images as the OCT instrument is progressed through the lumen, wherein successive en face images of a three-dimensional representation can be reconstructed (referred to as “pullback”).

However, the conventional longitudinal view of an OCT using pullback does not accurately represent the curvature of a lumen. Regardless of the curvature of the catheter, the longitudinal view represents the catheter as a straight segment, which when reconstructed, leads to a planar rendering. Because the catheter's curvature is not represented, this is translated to the lumen's true curvature not being represented in the longitudinal view.

The lack of an accurate representation of the lumen could result in poor stent placement due to lack of awareness of the lumen's curvature. By way of example: a lesion could be located in a highly tortuous region, but the longitudinal view will not represent the tortuosity. Without the guidance of angiogram or coregistration, this could result in lumen damage if a physician is not careful when placing the stent.

In addition, in some angiograms, the tortuosity of the lumen may be hidden if the tortuosity happens to be close to orthogonal to the viewing plane.

As a result, existing diagnostic and treatment procedures are imperfect, and since proper treatment requires positioning the catheter precisely and avoiding damage to the walls of the lumen, the lack of precise knowledge of a shape of a lumen renders much of the vasculature off-limits to existing procedures. Additionally, intravascular images and measurements can be distorted in counter-intuitive ways by catheter orientation. For example, where an intravascular imaging procedure shows a blood vessel wall on a computer monitor, a human viewer tends to interpret the image as though the imagined catheter and blood vessel are parallel and co-axial. Without information about the catheter position, and ultimate rendering of the lumen, the physician does not have enough information to perform the linear transformations to correct for distortions in the image.

Accordingly, it is particularly beneficial to devise apparatus, methods and systems for displaying three-dimensional images of a lumen which accurately account for and display curvatures of the lumen.

### SUMMARY

Thus, to address such exemplary needs in the industry, the presently disclosed apparatus teaches apparatus, systems and methods for employing the apparatus, including a medical device comprising: a bendable sheath having a hollow cavity extending the length of the bendable sheath; at least two markers configured about the bendable sheath a distance apart from one another; and an imaging core for determining a shape of the bendable sheath.

In various embodiments, the imaging core of the medical device is used for determining the shape of the bendable sheath by employing optical coherence tomography.

In other embodiments, the imaging core may be used for determining the shape of the bendable sheath by providing a three-dimensional shape of the bendable sheath.

In additional embodiments of the subject apparatus, the at least two markers have a different compressibility than the bendable sheath, when the sheath is bent.

In yet additional embodiments, the at least two markers are each perpendicular to a longitudinal direction of the bendable sheath and furthermore, the at least two markers may be parallel to one another.

The subject innovation further contemplates the bendable sheath being configured for manipulation into a tortuous cavity of a patient.

In further embodiments, the at least two markers are provided from the group consisting of an embedded particle into the sheath, doping of the sheath, etching of the sheath, photolithography of the sheath, alternatives thereof and combinations therefrom.

The subject innovation further teaches a method for providing a three-dimensional image of a medical device, comprising: providing a medical device comprising: a bendable sheath having a hollow cavity extending the length of the bendable sheath; at least two markers configured about the bendable sheath; and an imaging core for determining a shape of the bendable sheath, and determining a shape of the bendable sheath using the imaging core in a pullback procedure.

In further embodiments, the subject innovation teaches a system for amassing a three-dimensional image captured by a medical device, the system employing: a processor; a memory coupled to the processor, the memory having instructions for amassing the image, the instructions comprising: receiving data corresponding to the image; detecting at least two markers within the image; determining the three-dimensional position of the image based on the at least two markers; and amassing the three-dimensional image based on the determined position of the image.

The subject disclosure also teaches a computer-based method for manipulating an image which may be captured by a medical device, the method comprising the steps of: receiving data corresponding to the image; detecting a feature within the image; determining the center of the image based on the feature; and adjusting the image based on the determined center of the image.

These and other objects, features, and advantages of the present disclosure will become apparent upon reading the following detailed description of exemplary embodiments of the present disclosure, when taken in conjunction with the appended drawings, and provided paragraphs.

### BRIEF DESCRIPTION OF THE DRAWINGS

Further objects, features and advantages of the present disclosure will become apparent from the following detailed description when taken in conjunction with the accompanying figures showing illustrative embodiments of the present invention.

FIG. 1 is a cut-away side perspective view of an exemplary medical device, according to one or more embodiment of the subject apparatus, method or system.

FIG. 2 depicts a flattened rendering of the image in FIG. 1, to better detail various elements of the medical device, according to one or more embodiment of the subject apparatus, method or system.

FIG. 3 provides a front cut-away view of an exemplary medical device, according to one or more embodiments of the subject apparatus, method or system.

FIG. 4 depicts a cut-away side perspective view of an exemplary medical device, according to one or more embodiment of the subject apparatus, method or system.

Throughout the Figures, the same reference numerals and characters, unless otherwise stated, are used to denote like features, elements, components or portions of the illustrated embodiments. In addition, reference numeral(s) including by the designation “′” (e.g. 12′ or 24′) signify secondary elements and/or references of the same nature and/or kind. Moreover, while the subject disclosure will now be described in detail with reference to the Figures, it is done so in connection with the illustrative embodiments. It is intended that changes and modifications can be made to the described embodiments without departing from the true scope and spirit of the subject disclosure as defined by the appended paragraphs.

### DETAILED DESCRIPTION OF THE DISCLOSURE

Medical instruments such as endoscopic surgical instruments and catheters are well known and continue to gain acceptance in the medical field. The medical instruments generally include a flexible tube, commonly referred to as a sheath, as well as one or more tool channels that extend along (typically inside) the sheath to allow access to a target located at a distal end of the sheath.

The medical instruments are intended to provide flexible access within a patient, with at least one curve or more leading to the intended target.

In various advanced cases, the medical instrument may enhance maneuverability of the distal end of the instrument, utilizing robotized instruments (also referred to as “robots”) that control the distal portions of the medical instrument. In general, these robots are long instruments that are meant to be steerable through tortuous pathways and around objects to arrive at some desired location.

In these medical instruments, the sheath acts as a structured protective cover for the instrument and protects the surrounding tissue of the patient from unnecessary abrasions and trauma.

The subject innovation incorporates markers on the sheath that allow for accurate detection of the catheters' shape while using pullback, using OCT. As the shape of the sheath follows the shape of the lumen, the sheath incorporates a pattern to be used as markers to display a more accurate shape of the lumen. The markers are strategically placed about the sheath, typically positioned a distance apart from one another in the longitudinal direction of the sheath, to allow for consistent identification and ease of measurement.

The markers may have a different compressibility from the material in between the markers to allow for a different amount of flexibility in the sheath. This may be beneficial in that the different degree that the marker and non-marker material portions of the sheath may stretch in the outer and inner radius of a bend in the sheath window can be used to calculate the radius of the bend.

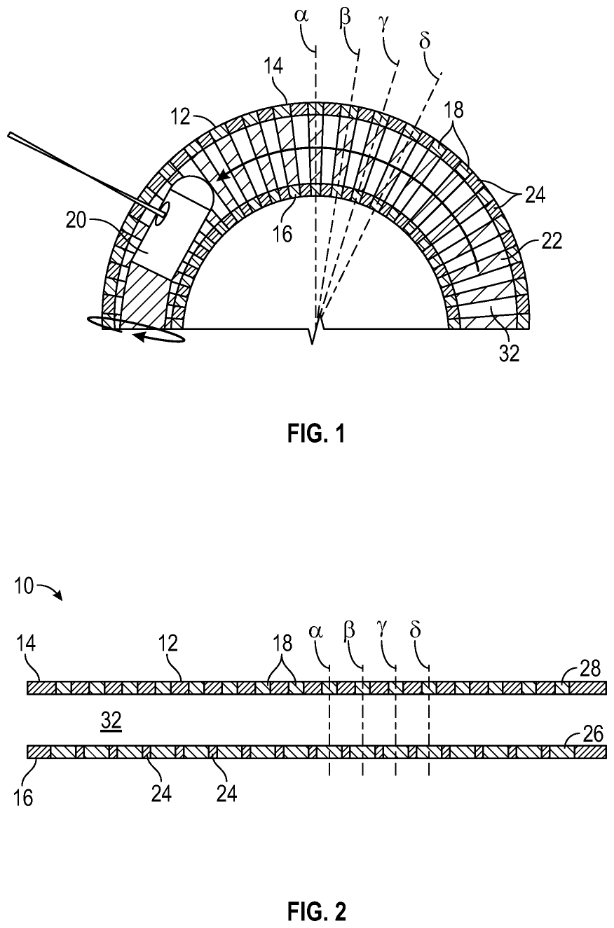

As depicted in FIG. 1, a cut-away side perspective view of a medical device 10, shown bent at an angle of about one-hundred and eighty degrees, is provided. The medical device 10 includes a bent sheath 12 having an outer radius 14 and an inner radius 16. Also depicted are the markers 18 provided within the sheath 12, which are arranged perpendicular to the longitudinal direction of the sheath 12. The sheath 12 has a central cavity 32, to accommodate a tool, here an OCT imaging core 20. In this embodiment, the markers 18 have a constant width at both the inner radius 16 and outer radius 14, allowing the non-marker sections 24 of the sheath 12, located between the markers 18, to compress and stretch to accommodate for the bent medical device 10. FIG. 1 further depicts the direction of the pullback 22 for the OCT imaging core 20, with the arrow signifying travel direction.

The typical OCT measuring core 20 measures the outer radius 14 as being equal in length to the inner radius 16, largely due to the planar measuring technique used in OCT. However, when the image is analyzed with the markers 18, the markers 18 provide the perspective necessary to account for the bend and identify the deviation between the inner radius 16 and outer radius 14, thus allowing for an accurate depiction of the bends in the sheath.

As seen in FIG. 2, the image of the sheath provided in FIG. 1 is flattened to better illustrate the difference between the inner radius 16 and outer radius 14. Here, we can see the stretch 26 and compression 28 in the non-marker sections 24 corresponding to the outer radius 14 and inner radius 16, respectively. Furthermore, as the markers 18 in this embodiment have a constant width, the unchanged width of the markers 18 can also be seen.

The ratio between the measured widths of the marker 18 in comparison with the measured widths of a non-marker sections 24 is used to calculate the angles (α, β, γ, δ) between adjacent markers 18, thus providing the data used to create a calibration curve to determine angle as a function of the ratio. Significantly, the markers 18 have no effect on image quality.

Although a constant width marker has been detailed herein, it is understood that a marker having a varying width is envisioned and has been used without limitation herein. As expected, the varying width would need to be taken into account for determining angle as a function of the ration between marker and non-marker sections, however, this is easily accomplished by calculating the compression and stretch in the markers based on the known deflection values for the material of the marker.

In another embodiment, the markers 18 may have flexibility in that the width of the markers 18 may vary. Accordingly, the markers 18 would have a different compression/stretch value than the non-marker section 24 of the sheath 12, to allow for detection of marker width, and calculation of the sheath angle.

Marker 18 width may be chosen so that it is sampled adequately by A-lines to get accurate measurement of width. Spacing between markers 18 may be chosen so that a three-dimensional angle can be measured accurately, while not hindering the bendability of the sheath. In addition, marker 18 period may be optimized so the helical path of OCT beam will fall on markers at multiple rotational angles to allow for more accurate determination of three-dimensional curvature, extending beyond one plane.

In various embodiments, the spacing between markers may be configured such that it may only allow detection of one marker at a time by the imaging core or other tool. Additional imaging modalities can be used with the sheath for sheath shape detection.

The markers may be made from embedded particles, metallic elements, doping the sheath, laser etching, and/or a photolithographic process.

Additional secondary markers may be included in the longitudinal direction for additional identification means and/or for combating non-uniform rotational distortion (NURD).

In addition, as the markers do not add to the overall size of the medical device, reduce the size of the tool channel, and do not affect the flexibility of the medical device, they may be incorporated into existing applications without any disadvantages.

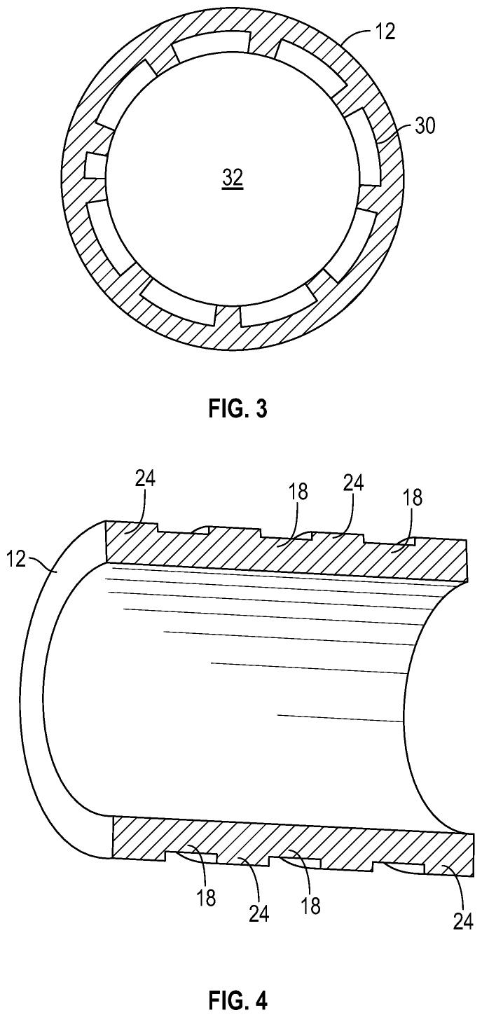

As provide in FIG. 3, additional markers may be configured along the longitudinal direction of the sheath (for instance—parallel to the longitudinal direction of the sheath) to detect and correct for NURD. As shown, the secondary markers 30 may be configured at a depth different than that of the markers 16, so as to distinguish one another, and avoid cross-detection.

FIG. 4 provides a cut-away image of a sheath 12, wherein the markers 18 are established by laser etching. The sheath 12 will have a different thickness along the window segment, creating marker sections 18 and non-marker sections 24. The markers 18 are compressed and stretched based on necessary curvature of sheath 12. As the thicker non-marker sections 24 will be more resistant to deformation than the thinner marker 18 section, angle calculations will need to be adjusted to account for the varying displacements between the two sections 18 and 24. Empirical data may be used to create a calibration curve to determine angle as a function of the ratio.

The subject apparatus, methods and systems provides images which may be displayed in various views, including: a longitudinal view; three-dimensional view; three-dimensional cross-sectional view; flythrough view; coregistered view in cooperation with an angiogram or other modality; real-time view; and combinations therefrom.

In addition, the subject innovation may be accompanied by complimentary or ancillary elements, including coregistration with an angiogram, as well as being used in other medical imaging modalities, including intravascular ultrasound. Using various computer-based algorithms, the captured images may be displayed in the longitudinal view, three-dimensional view, cross-sectional views, three-dimensional cross-sectional views, and flythrough views, to name a few.

Furthermore, the subject innovation may incorporate applications to apply machine learning, especially deep learning, to identify (e.g., detect, locate, or localize, etc.) a marker in the IVI imaging frame with greater or maximum success, and that use the results to perform coregistration more efficiently or with maximum efficiency. It is also a broad object of the present disclosure to provide IVI devices, systems, methods and storage mediums using an OCT, such as an interferometer (e.g., spectral-domain OCT (SD-OCT), swept-source OCT (SS-OCT), multimodal OCT (MM-OCT), etc.). Using artificial intelligence, for example, deep learning, one or more embodiments of the present disclosure may achieve a better or maximum success rate of marker detection from angiography data without (or with less) user interactions, and may reduce processing and/or prediction time to display coregistration result(s) based on the marker detection result(s).

In establishing an artificial intelligence modus, one or more architectural models (also referred to herein as “model(s)”) for the artificial intelligence method or algorithms, discussed herein, may be used. In one or more embodiments, a model may incorporate the locations of two endpoints of the major axis of the target marker in each image frame captured during OCT pullback. While architectural models discussed herein focus on a segmentation model, an object model (also referred to as a “regression model”, a regression model with residual learning, and a model that combines one or more features of the segmentation model and the regression model), the architectural models are not limited thereto. In one or more embodiments were a target marker may not be distinguishable yet from other markers, all of the markers may be marked as ground truths. In one or more embodiments where more data exists for training and/or a time series of frames (e.g., a video sequence) is utilized, a model may be improved to train a model with ground truth that includes only the target marker being masked. In one or more embodiments for a regression model or object model, a centroid of two edge locations may be considered as the ground truth location of a target marker in each image. In one or more embodiments, ground truth may be established, for example, by an expert graphically annotating the two marker locations in each frame of an angiographic image.

Selecting a model (e.g., segmentation model (classification model), regression model, regression model with residual learning, a combined model, etc.) may depend on a success rate of coregistration, which may be affected by a marker detection success rate, in the setting of a final application on validation/test data set(s). Such consideration(s) may be balanced with time (e.g., a predetermined time period, a desired time period, an available time period, a target time period, etc.) for processing/predicting and user interaction.

For the semantic segmentation model (also referred to as classification model or a segmentation model) architecture, one or more certain area(s) of an image are predicted to belong to one or more classes in one or more embodiments. There are many ways to set up the segmentation or classification model. By way of at least one example, a semantic segmentation may involve predicting a certain area of an image to one class or two classes. By way of a non-limiting, non-exhaustive embodiment example, the two classes may include whether a target (e.g., a pixel, an area of an image, a target object in an image, etc.) is a radiopaque marker (first class) or is not a marker (second class). In one or more output examples, each pixel may be assigned as a marker or not a marker. One or more embodiments of a semantic segmentation model may be performed using the One-Hundred Layers Tiramisu method discussed in “The One Hundred Layers Tiramisu: Fully Convolutional DenseNets for Semantic Segmentation” to Simon Jégou, et al., Montreal Institute for Learning Algorithms, published Oct. 31, 2017 (https://arxiv.org/pdf/1611.09326.pdf), which is incorporated by reference herein in its entirety. Convolutional Neural Networks (CNNs) may be used for one or more features of the present invention, including, but not limited to, artificial intelligence feature(s), detecting one or more markers, using the marker detection results to perform coregistration, image classification, semantic image segmentation, etc. For example, while other architectures may be employed, one or more embodiments may combine U-net and DenseNet features to perform semantic segmentation. U-net is a popular network architecture for two-dimensional (2D) image segmentation, and DenseNet has reliable and good feature extractors because of its compact internal representations and reduced feature redundancy. In one or more embodiments, a network may be trained by slicing the training dataset, and not down-sampling the data (in other words, image resolution may be preserved or maintained).

In one or more embodiments, the segmentation model with post-processing may be used with one or more features from “The One Hundred Layers Tiramisu: Fully Convolutional DenseNets for Semantic Segmentation” to Simon Jégou, et al., Montreal Institute for Learning Algorithms, published Oct. 31, 2017 (https://arxiv.org/pdf/1611.09326.pdf), which is incorporated by reference herein in its entirety.

For the object detection model (also referred to as the regression model as aforementioned) architecture, one or more embodiments may use an angiogram image or images as an input and may predict the marker location in a form of a spatial coordinate. This approach/architecture has advantages over semantic segmentation because the object detection model predicts the marker location directly, and may avoid post-processing in one or more embodiments. The object detection model may be created or built by using or combining convolutional layers, max-pooling layers, and/or fully-connected dense layers. Different combinations may be used to determine the best performance test result. The performance test result(s) may be compared with other model architecture test results to determine which architecture to use for a given application or applications. As aforementioned, in one or more embodiments, a convolutional autoencoder (CAE) may be used. In one or more embodiments, a support vector machine (SVM) classifier may be used (which, in one or more embodiments, may be a machine learning classifier that may be used for regression and classification), and reference data may be included to use as a reference standard for the SVM classifier. While SVM may involve two classes in an embodiment, multiple two class classifications may be performed for embodiments having two or more classes.

Furthermore, one or more embodiments may use one or more features for a regression model as discussed in “Deep Residual Learning for Image Recognition” to Kaiming He, et al., Microsoft Research, Dec. 10, 2015 (https://arxiv.org/pdf/1512.03385.pdf), which is incorporated by reference herein in its entirety.

In addition, One or more methods or algorithms for calculating stent expansion/underexpansion or apposition/malapposition may be used in one or more embodiments of the present disclosure, including, but not limited to, the expansion/underexpansion and apposition/malapposition methods or algorithms discussed in U.S. Pat. Pub. Nos. 2019/0102906 and 2019/0099080, which publications are incorporated by reference herein in their entireties. One or more methods or algorithms for calculating or evaluating cardiac motion using an angiography image and/or for displaying anatomical imaging may be used in one or more embodiments of the present disclosure, including, but not limited to, the methods or algorithms discussed in U.S. Pat. Pub. No. 2019/0029623 and U.S. Pat. Pub. No. 2018/0271614 and WO 2019/023382, which publications are incorporated by reference herein in their entireties.

Finally, one or more methods or algorithms for performing co-registration and/or imaging may be used in one or more embodiments of the present disclosure, including, but not limited to, the methods or algorithms discussed in U.S. Pat. App. No. 62/798,885, filed on Jan. 30, 2019, and discussed in U.S. Pat. Pub. No. 2019/0029624, which application(s) and publication(s) are incorporated by reference herein in their entireties.

1. A medical device comprising:
a bendable sheath having a hollow cavity extending the length of the bendable sheath;
at least two markers configured about the bendable sheath a distance apart from one another; and
an imaging core configured for movement in the hollow cavity, such that the imaging core can determine a shape of the bendable sheath.

2. The medical device of claim 1, wherein the imaging core for determining the shape of the bendable sheath employs optical coherence tomography.

3. The medical device of claim 1, wherein the imaging core for determining the shape of the bendable sheath provides a three-dimensional shape of the bendable sheath.

4. The medical device of claim 1, wherein the imaging core can determine a three-dimensional shape of the bendable sheath by a pullback procedure.

5. The medical device of claim 1, wherein the at least two markers have a different compressibility than the bendable sheath is bent.

6. The medical device of claim 1, wherein the at least two markers are each perpendicular to a longitudinal direction of the bendable sheath and the at least two markers are parallel to one another.

7. The medical device of claim 1, wherein the shape of the bendable sheath determined is a three-dimensional shape.

8. The medical device of claim 1, wherein the bendable sheath is configured for manipulation into a tortuous cavity.

9. The medical device of claim 1, wherein the at least two markers are provided from the group consisting of an embedded particle into the sheath, doping of the sheath, etching of the sheath, photolithography of the sheath, alternatives thereof and combinations therefrom.

10. A method for providing a three-dimensional image of a medical device, comprising:
providing a medical device comprising:
a bendable sheath having a hollow cavity extending the length of the bendable sheath;
at least two markers configured about the bendable sheath; and
an imaging core configured for movement in the hollow cavity, and for determining a shape of the bendable sheath, and
determining a shape of the bendable sheath using the imaging core in a pullback procedure.

11. The method of claim 8, wherein the imaging core for determining the shape of the bendable sheath employs optical coherence tomography.

12. The method of claim 8, wherein the imaging core for determining the shape of the bendable sheath provides a three-dimensional shape of the bendable sheath.

13. The method of claim 8, wherein the at least two markers have a different compressibility than the bendable sheath is bent.

14. The method of claim 8, wherein the at least two markers are each perpendicular to a longitudinal direction of the bendable sheath and the at least two markers are parallel to one another.

15. The method of claim 8, wherein the bendable sheath is configured for manipulation into a tortuous cavity of a patient.

16. The method of claim 8, wherein the at least two markers are provided from the group consisting of an embedded particle into the sheath, doping of the sheath, etching of the sheath, photolithography of the sheath, alternatives thereof and combinations therefrom.

17. The method of claim 8, wherein the shape of the bendable sheath determined is a three-dimensional shape.

18. A system for amassing an image captured by a medical device, the system employing:
a processor;
a memory coupled to the processor, the memory having instructions for amassing the image, the instructions comprising:
receiving data corresponding to the image;
detecting at least two markers within the image;
determining the position of the image based on the at least two markers; and
amassing the image based on the determined position of the image.

19. The system of claim 18, wherein the image is a three-dimensional image.

20. A non-transitory computer-readable storage medium storing at least one program for causing a computer to execute a method for training a model using artificial intelligence, the method comprising:
collecting or receiving data corresponding to the image;
detecting at least two markers within the image;
determining the position of the image based on the at least two markers;
deciding a model to be trained, including model architecture and parameters;
training a model with data corresponding to the image and evaluating the model;
determining whether the performance of the trained model is sufficient, and in the event that the trained model is not sufficient, repeating the deciding, the training, the estimating, and the determining, or, in the event that the trained model is sufficient, saving the trained model to a memory, and
amassing the image based on the trained model in the memory.
